(19) United States
(12) Patent Application Publication
(10) Pub. No.: US 2024/0350393 A1
(43) Pub. Date: Oct. 24, 2024

(54) AQUEOUS PERSONAL CARE COMPOSITIONS COMPRISING CARBOXYMETHYL CELLULOSE (CMC), PROCESS FOR PREPARING THE SAME AND USE THEREOF

(71) Applicant: ISP Investments LLC, Wilmington, DE (US)

(72) Inventors: MICHAEL FRANZKE, Barendrecht (NL); Lidia KULCSAR, Flanders, NJ (US); Carolie Claudine ALONSO, Breda (NL); Gijsbert KROON, Hardinxveld Giessendam (NL)

(73) Assignee: ISP Investments LLC, Wilmington, DE (US)

(21) Appl. No.: 18/683,162

(22) PCT Filed: Jul. 27, 2022
(86) PCT No.: PCT/US22/38489

§ 371 Date: Feb. 12, 2024

Related U.S. Application Data
(60) Provisional application No. 63/232,760, filed on Aug. 13, 2021.

(51) Int. Cl. A61K 8/73; A61Q 5/06

(52) U.S. Cl. CPC A61K 8/731; A61Q 5/06; A61K 2800/48; A61K 2800/594

(57) ABSTRACT

Disclosed is an aqueous personal care composition, particularly in hair care composition comprising 0.1 to 10 wt. % of low substituted carboxymethyl cellulose (CMC) alone or in combination with 0.01 to 10 wt. % of at least one natural or synthetic polymer, 0.01 to 60 wt. % of at least one additive; and water; wherein, the low substituted carboxymethyl cellulose has an average degree of carboxymethyl substitution (DS) per anhydroglucose unit is in the range of from about 0.5 to about <0.7.

### FIELD OF THE INVENTION

The present disclosure relates to an aqueous personal care composition, particularly hair care composition comprising carboxymethyl cellulose (CMC) having an optimized degree of substitution (DS) and process for preparing the same.

### BACKGROUND OF THE INVENTION

Conventionally, personal care products are used for improved moisturization, absorption, humidity resistance, curl retention, feel good factor or enhanced appearance. There are naturally derived technologies already on the market such as Xanthan Gum, Bentonite, Hectorite, cellulose gum and guar. The challenge though is that most of the technologies are providing only thickening but not suspension performance. The ingredients which suspend can have a negative influence on sensorial benefits (rheology, foam) and silicone deposition. Also, the stability can be an issue. Currently no single ingredients are meeting all the requirements.

Many natural and synthetic polymers have been used to improve rheology in personal care products. Viscosity and the degree of substitution (DS) of polymers in solutions determine their functionality. One such preferred choice of polymer for personal care applications is carboxymethyl cellulose (CMC).

U.S. Pat. No. 2,495,767 assigned to John David Reid and George C. Daul discloses continuous fibres spun from carboxymethyl cellulose having a DS between 0.5 and 1.0.

U.S. Pat. No. 4,525,585 assigned to Diacel Chemical Industries Ltd. discloses sodium carboxymethyl cellulose with average degree of substitution of 0.4 to 1.6.

U.S. Pat. No. 9,181,659 assigned to CP Kelco OY discloses carboxymethyl alkali solutions having a DS of less than about 0.9 for use in high solid paper coating and barrier materials.

US 2003/0035783 A1 describes use of superabsorbent polymers in hair care focus on crosslinked polyacrylates and crosslinked starches.

U.S. Pat. No. 7,488,470B2 discloses permanent deformation and/or shaping of a keratinous substance comprises an organic absorbing agent crosslinked sodium carboxymethylcellulose, a modified starch, or mixtures thereof

PCT application 2020164769 assigned to Henkel discloses cosmetic product that is suitable for temporarily shaping keratin fibers contains saccharose; hydroxypropyl methylcellulose and 0 to 3% by weight carboxymethyl starch.

However, lower the degree of substitution in CMC, less is the solubility in water. Higher the degree of substitution, less stable is the emulsion or suspensions prepared using CMC's. In such instances, emulsion stability and rheology behavior are jeopardized making the product less suitable for personal care application. Therefore, there is a need in the art for a personal care suspension having carboxymethyl cellulose with an optimized degree of substitution wherein solubility in aqueous solutions and stability are not compromised.

### SUMMARY OF THE INVENTION

The primary aspect of the present application is directed to an aqueous personal care composition comprising: (a) 0.05 to 10.0 wt. % of low substituted carboxymethyl cellulose (CMC) alone or in combination with 0.01 to 10.0 wt. % of at least one natural or synthetic polymer; (b) 0.01 to 60 wt. % of at least one personal care additive; and (c) water; wherein, the low substituted carboxymethyl cellulose has an average degree of carboxymethyl substitution (DS) per anhydroglucose unit, in the range of from about 0.5 to about less than (<) 0.7.

Another aspect of the present application provides a process for preparing an aqueous personal care composition, the process comprising: (a) considering powder particles comprising low substituted carboxymethyl cellulose (CMC) alone or in combination with at least one natural or synthetic polymer; (b) dispersing the polymer powder in water under continuous mixing; and (c) obtaining the homogenous aqueous composition.

Another aspect of the present application provides an aqueous hair care suspension composition comprising: (a) 0.05 to 10.0 wt. % of low substituted carboxymethyl cellulose (CMC) alone or in combination with 0.01 to 10.0 wt. % of at least one natural or synthetic polymer; (b) 0.01 to 60 wt. % of at least one hair care additive; and (c) water; wherein, the low substituted carboxymethyl cellulose has an average degree of carboxymethyl substitution (DS) per anhydroglucose unit, in the range of from about 0.5 to about less than (<) 0.7.

### BRIEF DESCRIPTION OF DRAWINGS

In addition to the cited advantages and objects of the disclosure, one or more descriptions of the disclosure briefly summarized can be added by reference to certain embodiments thereof which are illustrated in the appended drawings. These drawings form part of the specification. However, it is to be noted that the appended drawings illustrate preferred embodiments of the disclosure and therefore are not limiting in their scope.

FIG. 1 illustrates Scanning Electron Micrograph (SEM) pictures of comparative CMC sample and inventive CMC sample grades.

FIG. 2 illustrates viscosity data of comparative CMC vs. inventive CMC sample grades.

FIG. 3 illustrates suspension studies of comparative CMC vs. inventive CMC sample grades.

FIG. 4 illustrates viscosity data of comparative CMC vs. inventive CMC-Cetyl HEC combination grades.

FIG. 5 illustrates comparative CMC performance evaluated on hair tresses.

FIG. 6 and FIG. 7 illustrates comparative CMC performance evaluated on curl retention.

FIG. 8 illustrates comparative CMC vs. polyquaternium-69 performance evaluated on hair characteristics.

FIG. 9a and FIG. 9b illustrates comparative CMC vs. crosslinked polyacrylic acid performance evaluated on stress values.

FIG. 10 illustrates CMC in styling gels stable at pH.

FIG. 11 illustrates CMC evaluated on yield stress.

FIG. 12 illustrates comparative CMC performance evaluated on stiffness.

### DETAILED DESCRIPTION OF THE INVENTION

The technical solutions of the present disclosure will be described clearly and better understood in combination with specific embodiments below, but those skilled in the art will understand that embodiments described below are part of embodiments of the present disclosure but not all of them and are only used for illustration of the present disclosure and should not be considered as limiting the scope of the disclosure. If any specific condition or process are not indicated in the examples, it is to be understood that the conditions are conventionally used by the manufacturer and is commercially available.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by the context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

As used herein, the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “carboxymethyl cellulose” or “CMC” refers to both commercially available CMC products under the tradename “Blanose”, Aqualon, and in-house manufactured products.

As used herein, the term “cosmetically acceptable” refers to molecular entities regarded as safe, approved by regulatory body, listed in pharmacopoeia for use in topical contact with tissues (e.g., the hair) without undue toxicity, incompatibility, instability, irritation, allergic response, or the like. This term is not intended to limit the composition it describes as for use solely as a cosmetic (e.g., the composition may be used as a pharmaceutical).

As used herein, the term “degree of substitution (DS)” or “substituted degree” or “substitution degree” generally refers to carboxymethyl cellulose with the average number of carboxymethyl groups substituted per unit of anhydroglucose. Each anhydroglucose (β-glucopyranose) unit has three reactive (hydroxyl) groups, so theoretically DS value can be in the range from zero (cellulose itself) to three (fully substituted cellulose). The position and DS were measured using NMR by the identification of protons and carbon shift of methyl groups in the anhydroglucose unit and anhydride groups.

As used herein, the term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetra polymers, quaternary polymers, and homologues. The term “copolymer” refers to a polymer consisting essentially of two or more different types of monomers polymerized to obtain said copolymer.

As used herein, the term “personal care composition” and “cosmetics” refer to compositions intended for use on or in human body such as skin, hair, oral including those to alter the color and appearance of skin and hair.

As used herein, the term “viscosity of a solution” or “average viscosity” refers to measure of its resistance to gradual deformation by shear stress, which is due to intermolecular cohesive forces. These forces are affected by some factors such as concentration, temperature, or degree of substitution.

As used herein, the term ‘rheology’ refers to “the science or study of how things flow”, a requisite for personal care products.

As used herein, the term ‘xanthan gum’ refers to a polysaccharide gum industrially capable of producing a change in rheology of a liquid. Any commercially available xanthan gum may be used in the present application. The suitable commercial xanthan gum includes, but not limiting to Xanthan Gum®, (Kelzan® from Kelco) or Veegum® (from R. T. Vanderbilt).

As used herein, the term “keratin substrate” or “keratinous substrate” as used herein includes skin, nails and “keratin fibers”, and wherein the “keratin fibers” means hair on head, eyelashes, eyebrows and other mammalian bodily hair.

The term “hair care composition” refers to any composition intended for use on the human body for protection from harmful or undesirable radiation from the sun.

As used herein, the term “cosmetically acceptable excipient” means any ingredient/compound or mixture of ingredients/compounds or compositions that are typically employed to produce other desirable effects in personal care compositions.

In one non-limiting embodiment, the present disclosure is directed to an aqueous personal care composition comprising: (a) 0.05 to 10 wt. % of low substituted carboxymethyl cellulose (CMC) alone or in combination with 0.01 to 10 wt. % of at least one natural or synthetic polymer; (b) 0.01 to 60 wt. % of at least one personal care additive; and (c) water; wherein, the low substituted carboxymethyl cellulose has an average degree of carboxymethyl substitution (DS) per anhydroglucose unit, in the range of from about 0.5 to about <0.7.

CMC is a biodegradable, highly compatible anionic polymer obtained from cellulose (naturally sourced from cotton linters, wood pulp, or other cellulosic compounds) by substitution of at least a portion of the hydroxyl groups in cellulose molecule for carboxymethyl ether groups.

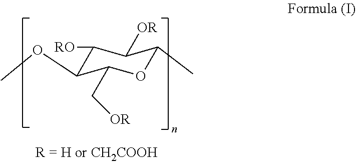

Carboxymethyl cellulose is commercially (CMC) obtained by alkalization reaction of cellulose with sodium mono chloroacetate and has degree of substitution (DS) in the range of 0.4 to 1.3. CMC is completely soluble at DS above 0.4 and hydro affinity of CMC increases with increasing DS, while this polymer is swellable but insoluble below 0.4. Present disclosure employs CMC having degree of substitution in the range of 0.5 to <0.7. Such carboxymethyl cellulose is herein referred to as “low substituted Carboxymethyl Cellulose (CMC)” or “carboxymethyl cellulose (CMC) with low degree of substitution”.

The carboxymethyl cellulose is present in a concentration range of from about 0.1 to about 10.0 wt. % of the total personal care composition. The natural or synthetic polymer is present in a concentration range of from about 0.01 to about 10.0 wt. % of the total personal care composition. The care composition additionally comprises 0.01 to 10 wt. % of care additive and water.

In another non-limiting embodiment, the low substituted carboxymethyl cellulose is present in the form of solid particulate or powder, having an average viscosity range of from about 2500 to about 20000 cps in 1% aqueous solution as measured by Brookfield, spindle A, speed 5.

Molecular weight of cellulose compounds is determined by GPC/SEC analysis. Gel Permeation Chromatography (GPC), also referred to as Size Exclusion Chromatography (SEC), is a valuable tool for the characterization of molecular weight, average molecular weight, and molecular weight distribution for polymers. For purposes of this invention, all molecular weights are given in weight average molecular weight (Mw) and measured in Daltons (Da). In an embodiment of this invention, the weight average molecular weight of the carboxymethyl cellulose will be in the range of from about 1000,000 to about 2000,000 Daltons. Molecular weight is one of the factors for differentiating CMC sample grades and the details are provided in Table 1.

Natural polymers used in the compositions of this invention can be selected from the group including, but not limited to xanthan gum, guar gum, carob gum, konjac gum, sclerotium gum, acacia gum, cellulose gum, pullulan, microcrystalline cellulose, Caesalpinia spinosa gum, carrageenan, dehydroxanthan gum, potato starch modified, glucomannan, magnesium aluminium silicate, bentonite, corn starch modified, polyester based polymer, tara gum, cassia gum, fenu greek, locust bean gum, pectin, starches and modified starches, alginate and alginic acid based polymers, hemi-celluloses and gellan gum. Preferably, natural polymer is xanthan gum. Xanthan is a polysaccharide with polyglucose as main polymer backbone with 3-unit acetylated side chains including glucose, glucuronic acid, typically present as a mixed potassium, sodium and calcium salt, and mannose residues. Xanthan polymers are typically obtained from bacterial fermentations, particularly of Xanthomonas campestris and related microorganisms.

Synthetic polymer useful in the practice of this invention include an acrylate based polymer, methacrylate based polymer, maleic anhydride based polymer, polyurethane based polymer, vinyl pyrrolidone based polymer vinylcaprolactam based polymer, vinylalcohol based polymer, vinylamide based polymer, vinylether based polymers and polylactic acid/polylactic acid salt or modified polysaccharide polymer.

Synthetic polymers useful in the practice of this invention include an acrylate based, methacrylate-based polymer, maleic anhydride based polymer, polyurethane based polymer or a vinyl pyrrolidone based polymer. Suitable acrylates or alkyl acrylates includes but not limited to (meth)acrylic acid, methyl (meth)acrylate, ethyl (meth)acrylate, propyl (meth)acrylate, isopropyl (meth)acrylate, butyl (meth)acrylate, amyl (meth)acrylate, isobutyl (meth)acrylate, t-butyl (meth)acrylate, pentyl (meth)acrylate, isoamyl (meth)acrylate, hexyl (meth)acrylate, heptyl (meth)acrylate, octyl (meth)acrylate, isooctyl (meth)acrylate, 2-ethylhexyl (meth)acrylate, nonyl (meth)acrylate, decyl (meth)acrylate, isodecyl (meth)acrylate, undecyl (meth)acrylate, dodecyl (meth)acrylate, lauryl (meth)acrylate, octadecyl (meth)acrylate, and stearyl (meth)acrylate; alkoxyalkyl (meth)acrylates, particularly d-alkoxy d-alkyl (meth)acrylates, such as butoxyethyl acrylate and ethoxyethoxyethyl acrylate; aryloxyalkyl (meth)acrylate, particularly aryloxy C4 alkyl (meth)acrylates, such as phenoxyethyl acrylate, single and multi-ring cyclic aromatic or non-aromatic acrylates such as cyclohexyl acrylate, benzyl acrylate, dicyclopentadiene acrylate, dicyclopentanyl acrylate, tricyclodecanyl acrylate, bornyl acrylate, isobornyl acrylate, alcohol-based (meth)acrylates such as polyethylene glycol monoacrylate, polypropylene glycol monoacrylate, methoxyethylene glycol acrylate, methoxypolypropylene glycol acrylate, methoxypolyethylene glycol acrylate, ethoxydiethylene glycol acrylate, and various alkoxylated alkylphenol acrylates such as ethoxylated (4) nonylphenol acrylate, amides of (meth)acrylic acid such as diacetone acrylamide, isobutoxymethyl acrylamide, t-octyl acrylamide. Suitable lactams include N-vinyl pyrrolidone, N-vinyl caprolactam, N-methyl-pyrrolidone, N-hydroxymethyl pyrrolidone, N-hydroxyethyl pyrrolidone, and N-hydroxypropyl pyrrolidone. Representative anhydrides include formic anhydride, succinic anhydride, maleic anhydride and acetic anhydride. Preferred synthetic polymers include methyl vinyl ether/maleic anhydride copolymer crosslinked with decadiene and polyacrylic acid.

The modified polysaccharide polymers useful in the practice of this invention include an methyl, hydroxyethyl, hydroxypropyl, carboxymethyl, sulfonate, phosphate, trimethylamine, hydroxypropyl, propyl groups of xanthan gum, guar gum, carob gum, konjac gum, sclerotium gum, acacia gum, cellulose gum, pullulan, microcrystalline cellulose, Caesalpinia spinosa gum, carrageenan, dehydroxanthan gum, potato starch modified, glucomannan, magnesium aluminium silicate, bentonite, corn starch modified, polyester based polymer, tara gum, cassia gum, fenu greek, locust bean gum, pectine, starches and modified starches, alginate and alginic acid based polymers, hemi-celluloses and gellan gum.

In another non-limiting embodiment, the current personal care composition is formulated into a hair-care product, a shampoo, a hair conditioner, leave in and rinse off conditioner, a styling hair composition, a hair perming product, a hair relaxant, a hair straightener, a hair spray and lacquer, a permanent hair dyeing system, a hair styling mousse, a hair gel, a semi-permanent hair dyeing system, a temporary hair dyeing system, a hair bleaching system, a permanent hair wave system, a hair setting formulation, a liquid soap, a bar soap, a fragrance and/or odoriferous ingredients consisting preparation, a deodorizing and antiperspirant preparation, a body oil, a body lotion, a body gel, a treatment cream, a body cleaning product, a skin protection ointment, a shaving and aftershave preparation, a skin powder, a lipstick, a foundation, a nail varnish, an eye shadow, a mascara, a dry and moist make-up, a blush or a blusher, a powder, a depilatory agent, a leave-on skin lotion and cream, a shower gel, a toilet bar, a sunless tanner, a sunscreen lotion, a sunscreen spray, a sunscreen cream, a hair styling gel, a serum, a mask and a tooth paste.

In another non-limiting embodiment, the low substituted CMC alone or in combination is used in the preparation of personal care composition in the form of an emulsion, a dispersion, a suspension, a lotion, a cream, a foam, a spray, a gel, a soap bar, a stick, a mask, a pad or a patch.

In another non-limiting embodiment, the present disclosure is directed to an aqueous hair care composition, the natural or synthetic polymer is a dihydroxypropyl substituted cassia gum (DHP-CG polymer), a cationic guar, a hydroxy ethyl cellulose (HEC), a methylhydroxypropylcellulose (MHPC) or a sorbitan oleate decylglucoside cross-polymer.

In another non-limiting embodiment, the present disclosure is directed to an aqueous hair care composition is formulated as a spray, a lotion, a mousse, a fluid, a serum, a solution, a perm, an emulsion, a gel, a vesicle a dispersion, a paste, a cream, a solid stick, a shampoo, a balm, a wipe, a milk, a foam, a jelly, a pomade, a wax, a powder, a bar or a liquid.

In another non-limiting embodiment, the present disclosure is directed to an aqueous hair care composition comprising the hair care additives selected from the group consisting of water-insoluble ingredients, oxidizing agents, conditioning agents, humectants, pH adjusting buffers, waxes, mineral oils, emulsifiers, fragrances, preservatives, fatty substances, gelling agents, thickeners, emollients, hydrophilic or lipophilic active agent, antioxidants, sequestering agents, preserving agents, acidifying or basifying agents, fragrances, fillers, dyestuffs, plant extracts, moisturizers, proteins, peptides, neutralizing agents, solvents, anti-dandruff ingredients, reducing agents, silicones, ceraphyl esters and combinations thereof.

In another non-limiting embodiment, the present disclosure is directed to an aqueous hair care composition comprising the hair care additives consisting of oils selected from the group consisting of coconut oil, jojoba oil almond oil, olive oil. lavender oil, aloe vera oil, tea tree oil, grapeseed oil, safflower seed oil, argan oil, avocado oil, rosemary oil, cederwood oil, sage oil, arnica oil, eucalyptus oil, castor oil, thyme oil, rosemary oil, chamomile oil, macadamia nut oil, kukui nut oil, sunflower seed oil, sesame seed oil, rice bran oil, flax seed oil, soybean oil, pomegranate oil, peppermint oil, spearmint oil, clary sage oil and mixtures thereof.

According to one embodiment of the present application, the hair care composition comprises any silicones which are known to a person skilled in the art, such silicones may be present in the form of oils, waxes, resins, or gums. Silicones for the present invention can be selected from Encyclopedia of Polymer Science and Engineering, vol. 15, 2d ed., pp 204-308, John Wiley & Sons, Inc. (1989), is incorporated herein by reference. Non-limiting examples of suitable silicone conditioning agents, and optional suspending agents for the silicone, are described in detail in U.S. Reissue Pat. No. 34584, U.S. Pat. Nos. 5,104,646, and 5,106,609 and can be readily referred for the purposes of the invention.

Suitable silicones for the present application are duly disclosed in U.S. Pat. Nos. 2,826,551, 3,964,500, 4,364,837, British Patent No. 849433, EP-A-0 582152, WO 93/23009, and Silicon Compounds, Petrarch Systems, Inc. (1984), all of which are incorporated herein by reference.

Suitable silicones for the present application selected from the group consisting of silicone fluids (oils), silicone elastomers, silicone resins, polysiloxanes, linear silicones, branched silicones, cyclic silicones, aminosilicones, amodimethicone, dimethicones, dimethiconols, silicone polyethers, silicone quaternium compounds and mixtures thereof.

The suitable silicones of the present application would include but are not limited to polyorganosiloxanes, polyalkyl siloxanes, polyaryl siloxanes, polyalkyl aryl siloxanes, silicone gums and resins, and polyorgano siloxanes modified by organofunctional groups, and mixtures thereof. Suitable polyalkyl siloxanes include polydimethyl siloxanes with terminal trimethyl silyl groups or terminal dimethyl silanol groups (dimethiconol) and polyalkyl (C1-C25) siloxanes. Suitable polyalkyl aryl siloxanes include polydimethyl methyl phenyl siloxanes and polydimethyl diphenyl siloxanes, linear or branched. The silicone gums suitable for use herein include polydiorganosiloxanes preferably having a number-average molecular weight between 200,000 and 1,000,000, used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxanes. Suitable silicone resins include silicones with a dimethyl/trimethyl siloxane structure and resins of the trimethyl siloxysilicate type. The organo-modified silicones suitable for use in the invention include silicones such as those previously defined and containing one or more organofunctional groups attached by means of a hydrocarbon radical and grafted siliconated polymers. Particularly preferred are amino functional silicones. The silicones may be used in the form of emulsions, nano-emulsions, or micro-emulsions.

In another non-limiting embodiment, the present disclosure is directed to an aqueous hair care composition comprising the hair care additive consisting of silicones selected from the group consisting of silicone fluids (oils), silicone elastomers, silicone resins, polysiloxanes, linear silicones, branched silicones, cyclic silicones, aminosilicones, amodimethicone, dimethicones, dimethiconols, silicone polyethers, silicone quaternium compounds and mixtures thereof.

In another non-limiting embodiment, the present disclosure is directed to an aqueous hair care composition that provides fixing or treating hair imparting properties comprising detangling, wet combability, wet feel, dry combability, dry feel, sheen, static flyaway control, humidity resistance, hydrophobicity, surface smoothening, no build-up, color, high humidity curl retention, strong hold, and split-end repair.

The desired pH of the hair care composition is in the range of from about 3 to about 13, and in some embodiment, it is preferably between about 4 to about 8. The utility levels of the pH modifying agent may be present in an effective amount required to achieve the desired pH level.

In some embodiments, the suitable range of low substituted carboxymethyl cellulose present in the personal care composition can be varied from about 0.05 wt. % to about 0.1 wt. %, from about 0.1 wt. % to about 1 wt. %, or from about 1 wt. % to about 2.5 wt. %, or from about 2.5 wt. % to about 5 wt. % or from about 5 wt. % to about 10 wt. %, based on the total weight of the aqueous personal care composition.

In some embodiments, the suitable range of low substituted carboxymethyl cellulose present in the hair care composition can be varied from about 0.05 wt. % to about 0.1 wt. %, from about 0.1 wt. % to about 1 wt. %, or from about 1 wt. % to about 2.5 wt. %, or from about 2.5 wt. % to about 5 wt. % or from about 5 wt. % to about 10 wt. %, based on the total weight of the aqueous hair care composition.

In some embodiments, the suitable range of natural or synthetic polymer present in the personal care composition can be varied from about 0.01 wt. % to about 0.05 wt. %, from about 0.05 wt. % to about 0.1 wt. %, from about 0.1 wt. % to about 1 wt. %, or from about 1 wt. % to about 2.5 wt. %, or from about 2.5 wt. % to about 5 wt. % or from about 5 wt. % to about 10 wt. %, based on the total weight of the aqueous hair care composition. In some embodiments, the suitable range of natural or synthetic polymer present in the hair care composition can be varied from about 0.01 wt. % to about 0.05 wt. %, from about 0.05 wt. % to about 0.1 wt. %, from about 0.1 wt. % to about 1 wt. %, or from about 1 wt. % to about 2.5 wt. %, or from about 2.5 wt. % to about 5 wt. % or from about 5 wt. % to about 10 wt. %, based on the total weight of the aqueous personal care composition.

In some embodiments, the suitable range of personal care ingredient present in the hair care composition can be varied from about 0.01 wt. % to about 0.05 wt. %, from about 0.05 wt. % to about 0.1 wt. %, from about 0.1 wt. % to about 1 wt. %, or from about 1 wt. % to about 2.5 wt. %, or from about 2.5 wt. % to about 5 wt. %, from about 5 wt. % to about 10 wt. %, from about 10 wt. % to about 15 wt. %, or from about 15 wt. % to about 20 wt. %, from about 20 wt. % to about 25 wt. %, from about 25 wt. % to about 30 wt. %, or from about 30 wt. % to about 35 wt. %, from about 35 wt. % to about 40 wt. %, from about 45 wt. % to about 50 wt. %, or from about 50 wt. % to about 55 wt. %, or from about 55 wt. % to about 60 wt. %, based on the total weight of the aqueous hair care composition.

In some embodiments, the suitable range of hair care ingredient present in the hair care composition can be varied from about 0.01 wt. % to about 0.05 wt. %, from about 0.05 wt. % to about 0.1 wt. %, from about 0.1 wt. % to about 1 wt. %, or from about 1 wt. % to about 2.5 wt. %, or from about 2.5 wt. % to about 5 wt. %, from about 5 wt. % to about 10 wt. %, from about 10 wt. % to about 15 wt. %, or from about 15 wt. % to about 20 wt. %, from about 20 wt. % to about 25 wt. %, from about 25 wt. % to about 30 wt. %, or from about 30 wt. % to about 35 wt. %, from about 35 wt. % to about 40 wt. %, from about 45 wt. % to about 50 wt. %, or from about 50 wt. % to about 55 wt. %, or from about 55 wt. % to about 60 wt. %, based on the total weight of the aqueous hair care composition.

In some embodiments, the suitable range of low substituted carboxymethyl cellulose has an average degree of carboxymethyl substitution (DS) per anhydroglucose unit present in the personal care composition can be varied from the is in the range of from about 0.5 wt. % to about 0.55 wt. %, from about 0.55 wt. % to about 0.6 wt. %, from about 0.6 wt. % to about 0.65 wt. %, or from about 0.65 wt. % to about 0.7 wt. % based on the total weight of the aqueous personal care composition.

In some embodiments, the suitable range of low substituted carboxymethyl cellulose has an average degree of carboxymethyl substitution (DS) per anhydroglucose unit present in the hair care composition can be varied from the is in the range of from about 0.5 wt. % to about 0.55 wt. %, from about 0.55 wt. % to about 0.6 wt. %, from about 0.6 wt. % to about 0.65 wt. %, or from about 0.65 wt. % to about 0.7 wt. %, based on the total weight of the aqueous hair care composition.

The compositions described herein have one or multiple benefits including improved sensory attributes, delivery of high viscosity, enhanced suspension capabilities, CMC having high degree of substitution, making it a preferred choice for the formulators. Excellent results have been observed for hair care applications.

Further, certain aspects of the present application are illustrated in detail by way of the following examples. The examples are given herein for illustration of the application and are not intended to be limiting thereof.

### EXAMPLES

### Example 1: Carboxymethyl Cellulose Sample Grades

TABLE 1
CMC Sample Grades

| | Viscosity | Molecular | |
|---|---|---|---|
| | @1% in water | Weight | Degree of |
| CMC Grade | (D15 speed 5) cps | (Daltons) | Substitution |
| Sample Grade 1 | 8080 | 1640000 | 0.5-0.6 |
| Sample Grade 2 | 15600 | 1440000 | 0.5-0.6 |
| Sample Grade 3 | 15200 | 1060000 | 0.5-0.6 |
| Commercial CMC | 2250 | 1060000 | 0.7-0.8 |

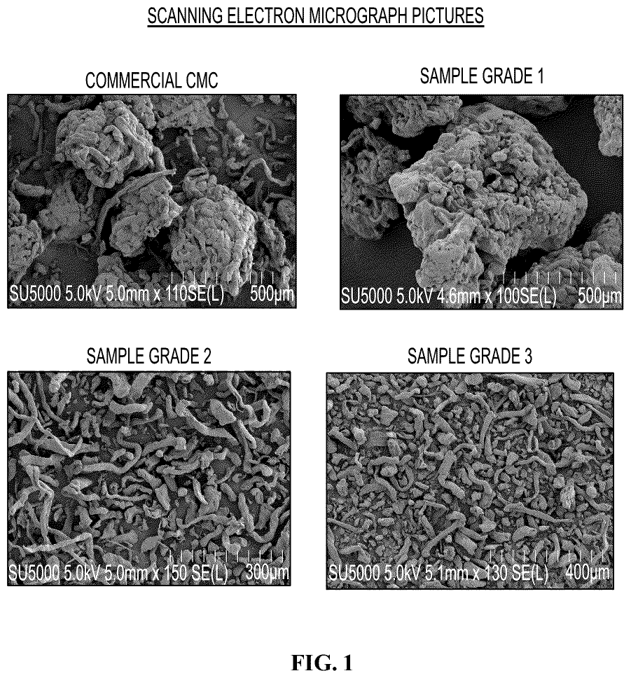

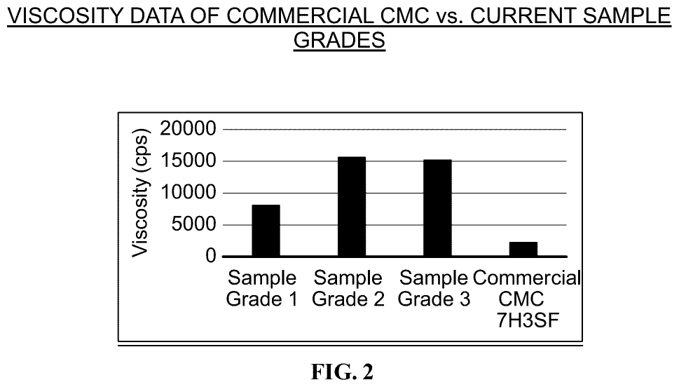

Table 1 shows carboxymethyl cellulose grades having different viscosities, molecular weights and degree of substitution were used for emulsion and suspension preparations (FIG. 1 and FIG. 2).

### Example 2: Aqueous Suspension Composition

TABLE 2
Aqueous Suspension Composition

| | |
|---|---|
| | Quantity |
| Ingredients | (wt. %) |

Phase A

| | |
|---|---|
| Purified Water | 85.9 |
| Sample Grade 1 or 2 (Cellulose Gum) | 1.5 |

Phase B

| | |
|---|---|
| Raspberry Ketone | 0.5 |
| 1,2-Hexanediol | 3.0 |

Phase C

| | |
|---|---|
| Water, glycerin, sodium PCA, Erythritol, | 3.0 |
| chondrus crispus (carrageenan), Xanthan Gum | |

Phase D

| | |
|---|---|
| Aloe Barbadensis leaf juice powder | 0.1 |
| Water | 1.0 |

Phase E

| | |
|---|---|
| Captivates GL or HC | 5.0 |
| Total | 100 |

### Example 3: Results of Suspension Stability Studies

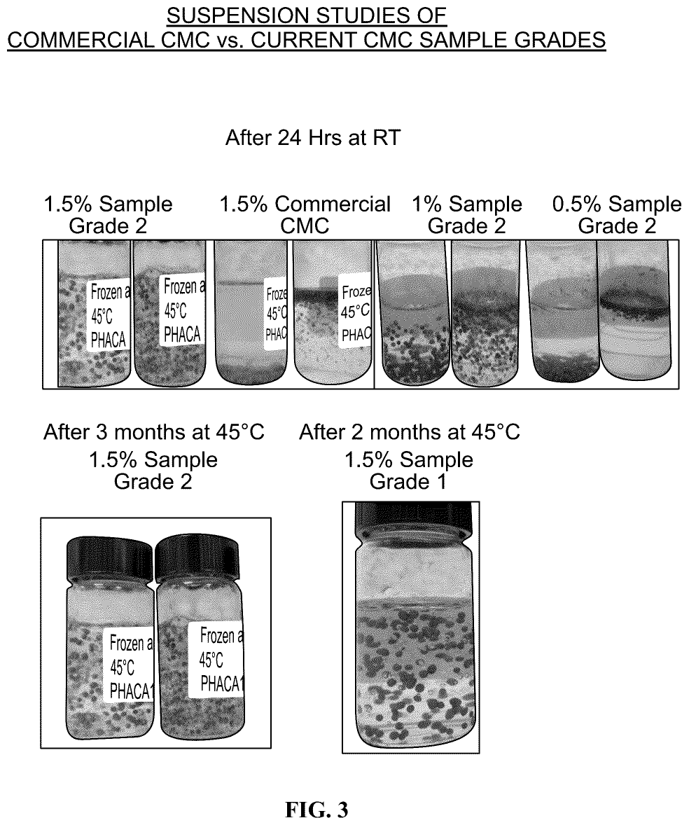

Aqueous suspension described in Example 2 was subjected to suspension studies. The results are shown in FIG. 3. It is observed that 1.5% of commercial CMC, lower amounts of current CMC samples grades (0.5% and 1.0%) either remain at the bottom or on the top of the solution and do not suspend for more than 24 hours at RT. The current sample grades even at higher concentrations (1.5%) remain suspended for three months at 45° C. Concentration of CMC determines the suspension stability.

CMC grades were subjected to stability studies using various emulsifiers and compared with commercial CMC sample with cetyl hydroxyethyl cellulose combination. (FIG. 4).

### Example 4: Carboxmethylcellulose on Hair Tresses

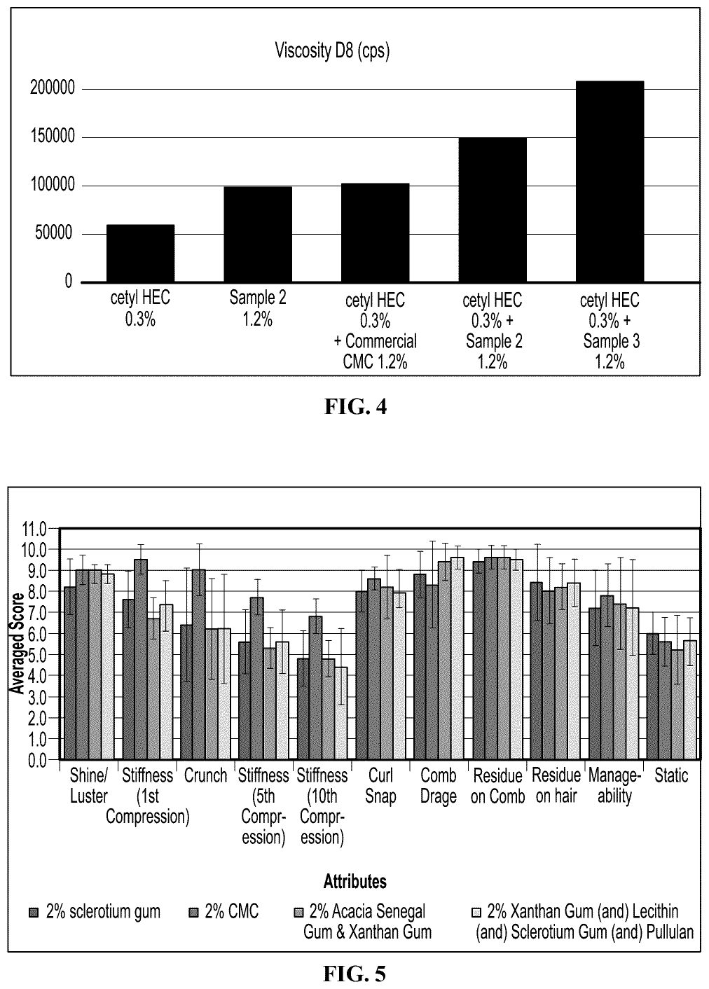

Carboxmethylcellulose, provides viscosity, humidity resistance, sensory and styling benefits to hair care products. In styling applications, CMC can be used as both a rheology modifier and styling polymer in one, without the need for separate viscosity and styling polymers. In styling applications, solutions of CMC form clear, brittle film without tack. Compared to other nature derived polymers used in styling applications, CMC outperforms these polymers in several key attributes—humidity resistance, stiffness and curl flexibility, as shown below. The higher the attribute score, the better the performance evaluated on hair tresses (FIG. 5)

2% aqueous solution of polymer (FIG. 5) was applied on damp 6 inch long/0.75-inch-wide virgin brown hair tresses, rolled into a curl on medium-sized hair roller, and dried in 45° C. oven for 4 hours. After drying, the hair was removed from the rollers, and evaluated by trained panelists on selected attributes.

### Example 5: Curl Retention Test

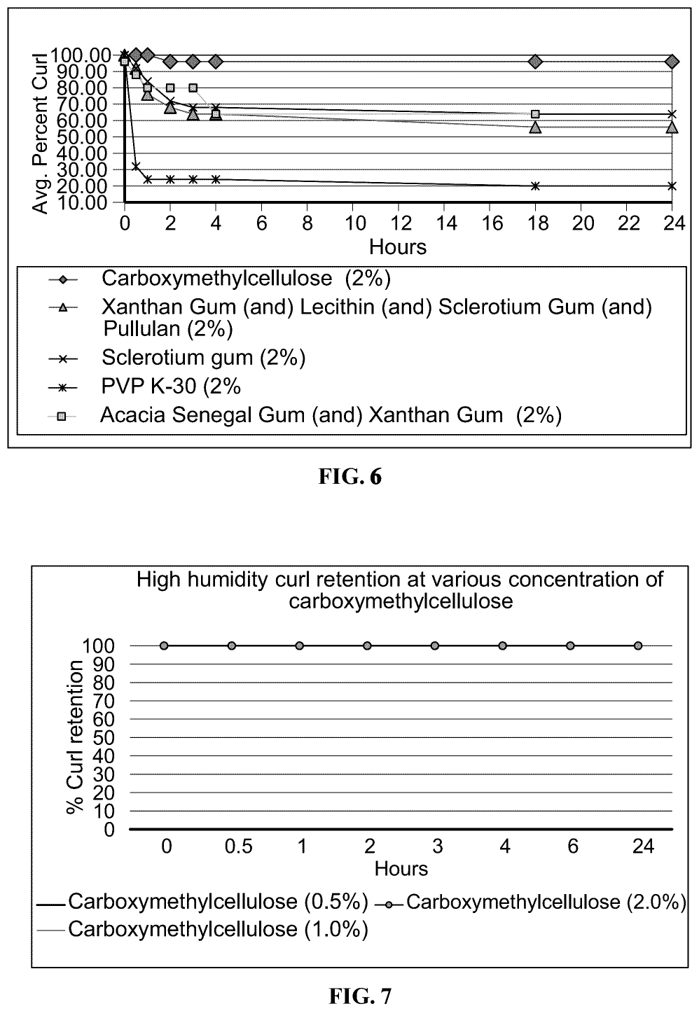

High humidity curl retention test (90% relative humidity, 80 F, 24 hours), CMC was outperformed nature derived polymers, providing significantly better humidity resistance (FIG. 6).

The CMC provides excellent humidity resistance even at low levels. Polymer was tested at 0.5%, 1% and 2% solids, and at all solid level maintained a 100% curl retention (FIG. 7).

### Comparative Example 6

Compared to traditional synthetic styling polymers (acetlylenics, acrylates, vinyl pyrrolidone), CMC has very similar styling performance—as seen in table below. Styling performance was compared to Polyquaternium-69 (FIG. 8).

### Example 7: CMC Sheer Thinning Viscosity

As a rheology modifier, CMC provides sheer thinning viscosity. Viscosity is also dose dependent—higher amounts of CMC provides higher viscosity. Viscosity was taken on aqueous solutions of the carboxymethylcellulose, with Brookfield RVT viscometer @ 10 rpm.

TABLE 3
CMC sheer thinning viscosity

| | 2% CMC | 1% CMC | 0.5% CMC |
|---|---|---|---|
| pH | 6.91 | 7.05 | 7.15 |
| Viscosity (TC@10) | 55000 | 6700 | 3100 |
| Appearance | Clear | Clear | clear |

### Comparative Example 8: CMC and Crosslinked Polyacrylic Acid-Stress Values

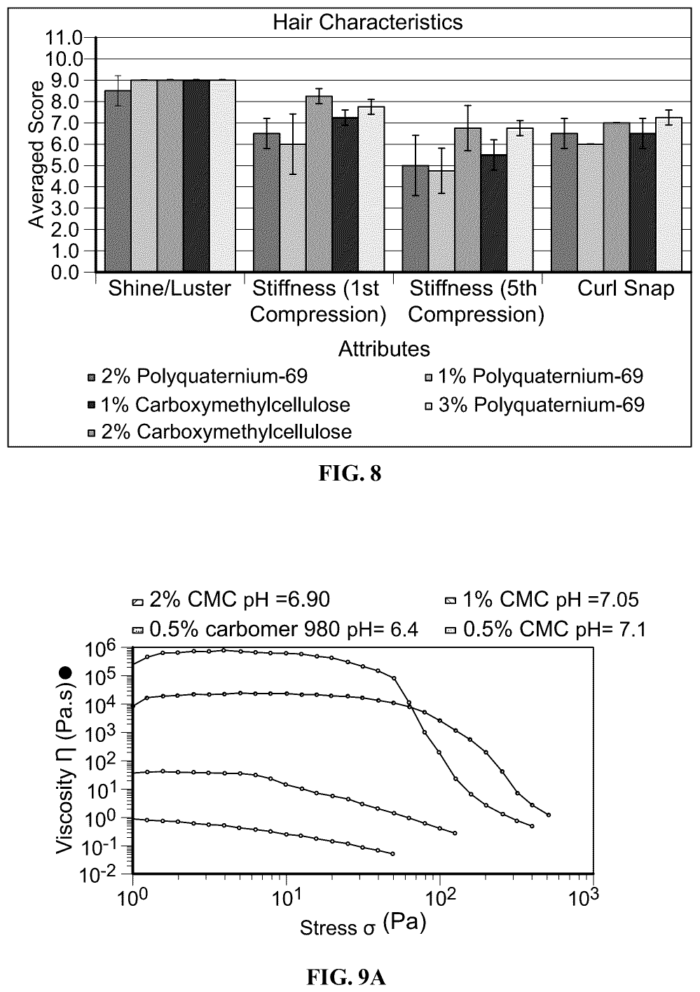

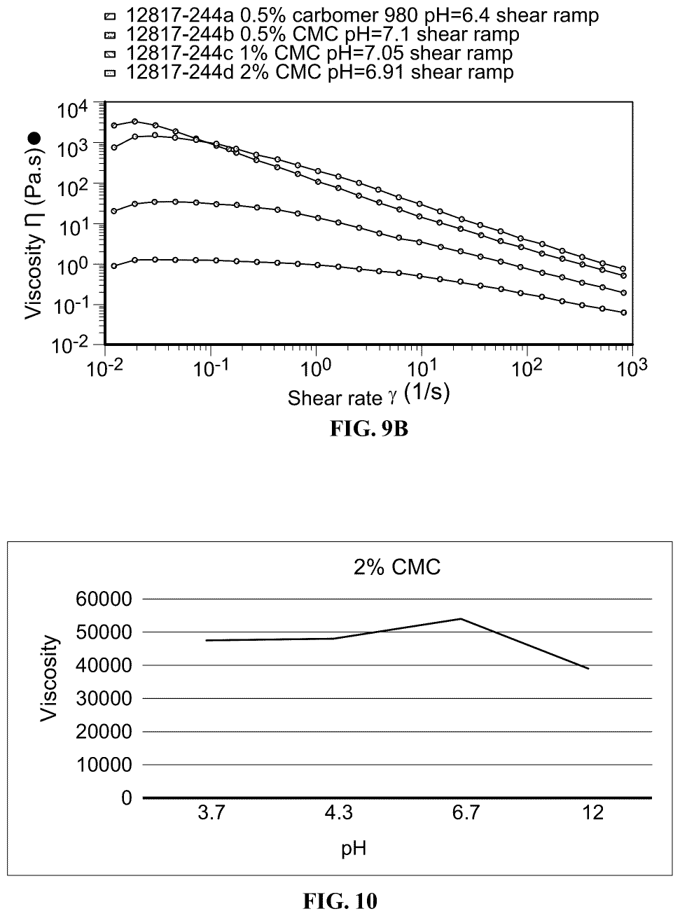

At 2% solids, CMC was a suitable replacement for acrylate-based thickeners, offering a nature derived, more eco-friendly alternative to commonly used acrylate rheology modifiers such as carbomer, acrylate copolymers, etc. Overall, CMC has lower zero shear viscosity than carbomer, but at 2% solids, it has a higher yield stress value. Higher yield stress values correlate with better suspension of oils, particulates, beads, etc., (FIG. 9a and FIG. 9b). A stress-controlled AR-G2 rheometer from TA instruments coupled with 40 mm sand blasted stainless steel parallel plates were used to measure yield stress and shear viscosity of the samples at 25° C.

TABLE 4
CMC sheer thinning viscosity

| Sample info | Yield stress, Pa | ZSV, Pa · s |
|---|---|---|
| 0.5% Carbomer 980 at pH 6.4 | 39 | 647890 |
| 2% CMC at pH = 7.1 | 89 | 23655 |
| 1% CMC at pH = 7.05 | 6.7 | 38 |
| 0.5% CMC at pH = 6.91 | NA | NA |

### Example 9: CMC in Styling Gels Stable at pH

CMC in styling gels is that the viscosity was stable under a wide pH range. Typical acrylate thickeners are salt sensitive and have a narrow functional pH range. As seen in FIG. 10, the viscosity of the CMC remains stable in pH range of 3-12.

### Example 10: CMC-Yield Stress

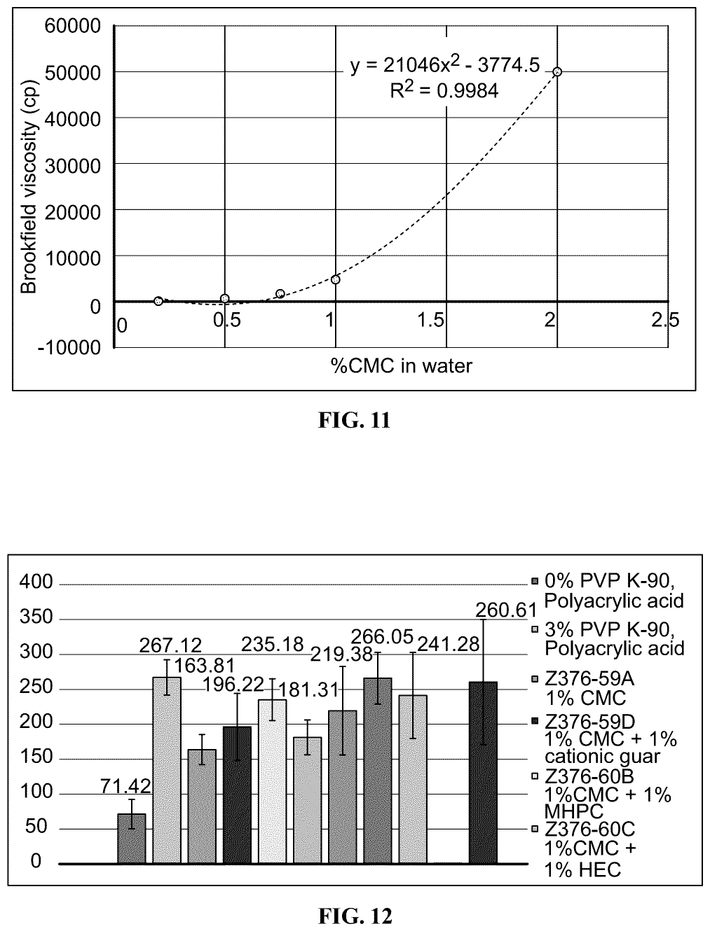

The Yield stress attained at from 1% CMC in water where in there is no yield stress for CMC at 0.5 and 0.75. Polynomoal viscosity build and yield stress attained at from 1% CMC in water shown in FIG. 11.

TABLE 5
CMC solutions

| | Brookfield | | Spindle - | | |
|---|---|---|---|---|---|
| CMC % | viscosity (cp) | | speed (rpm) | pH | % T |
| 0.2 | 127 | cp | #62-30 | 6.6 | 94 |
| 0.5 | 660 | cp | #62-30 | 6.5 | 99 |
| 0.75 | 1720 | cp | #63-30 | 6.6 | 98 |
| 1 | 4800 | cp | #64-30 | 5.4 (adjusted) | n.a. |
| 2 | 50 000 | cp | #07-30 | 5.1 (adjusted) | n.a. |

TABLE 6
CMC solutions- Yield Stress

| | | Cross over point | Yield |
|---|---|---|---|
| % CMC | Yield stress | (G′ = G″)/Flow point | stress G′ |
| 0.5 | No | — | — |
| 0.75 | No | — | — |
| 1.0 | yes | 16.34 Pa | 17.21 Pa |

### Example 11: CMC Combined With DHP Cassia

Process 1: Blend dry powder of CMC and DHP Cassia. and then add the powder into water phase resulting low viscosity and phase separated after 10 minutes.

Process 2: Solubilized CMC then add DHP Cassia results CSP destroyed viscosity and phase separated after 10 minutes. Due to high charge density of DHP Cassia, difficult to combine with CMC, tried at lower Cassia Hydroxypropyltrimonium Chloride level.

### Example 12: CMC Combined With Polymers

% CMC combined with 1% Cationic guar has suspending property.

TABLE 7
CMC combined with Polymers

| | Brookfield | #Spindle | | |
|---|---|---|---|---|
| | viscosity | speed | | |
| Combination | (cp) | (rpm) | pH | Comment |
| 1% CMC | 4800 | #64-30 | — | — |
| 0.5% CMC + 1% Cationic guar | <100 | | 5.3 | 1 to 2 ratios reduced dramatically viscosity and |
| | | | | remain insoluble particles in solution |
| 1% CMC + 2% Cationic guar | 3459 | #64-30 | 4.8 | 1 to 2 ratio reduced viscosity and remain insoluble |
| | | | | particles in solution |
| 1% CMC + 1% Cationic guar | 7800 | #64-30 | 4.9 | Build-up viscosity and suspended air bubbles. air |
| | | | | bubbles remain stable after 3 days observed. |
| 1% CMC + 1% Cationic guar + | 23500 | #07-30 | 4.8 | Build-up viscosity and suspended air bubbles. air |
| 0.5% MHPC | | | | bubbles remain stable after 3 days observed. |
| 1% CMC + 1% Cationic guar + | 20000 | #07-30 | 4.7 | Build-up viscosity and suspended air bubbles. air |
| 0.5% HEC | | | | bubbles remain stable after 3 days observed. |

### Example 13: CMC Combined With Polymers-Rheology Profile-Amplitude Sweep-Yield Stress

TABLE 8
Rheology profile - Amplitude sweep - Yield stress

| | | Cross over point | |
|---|---|---|---|
| | Yield | (G′ = G″)/ | Yield |
| Sample | stress | Flow stress | stress G′ |
| 1% CMC | yes | 16.34 Pa | 17.21 Pa |
| 1% CMC + 1% Cationic Guar | yes | 27.24 Pa | 50.48 Pa |
| 1% CMC + 1% Cationic Guar + | yes | 72.58 Pa | 125.1 Pa |
| 0.5% MHPC | | | |
| 1% CMC + 1% Cationic Guar + | yes | 65.11 Pa | 119.7 Pa |
| 0.5% HEC | | | |

### Example 14: Sensory Assessment on Caucasian Hair Tresses and Stiffness

TABLE 9
Sensory assessment on Caucasian hair tresses

| | Dry application | Wet application |
|---|---|---|
| Combination | (1 g product per 3 g hair) | (1 g product per 3 g hair) |
| 1.% CMC | Smooth, compact, and very | Easy to distribute, hair feel |
| | bouncy curl. No residue | clean, shine, bouncy. No |
| | | residue. |
| 1% CMC + 1% Cationic | Best initial hold, very | Easy to distribute, easy to |
| Guar | bouncy curl. No residue | style. Little bit residue left. |
| 1% CMC + 1% Cationic | Stiffest. No residue | Stiffest. Little bit residue |
| Guar + 0.5% MHPC | | left. |
| 1% CMC + 1% Cationic | Little lumpy structure. Dry | Easy to style. No residue |
| Guar + 0.5% HEC | fast. No residue | |

### Example 15: Gel-Cream

Formula 1% CMC+1% MHPC shows viscosity of 40.000 cps. Due to high viscosity, high yield stress and low oil level, it is difficult to conclude about stability. Hence, 3% of oil mix and 15% of sorbitan oleate decylglucoside crosspolymer (HLB 8-10) is added to reduce emulsifier level to 3% and 6%. (FIG. 12)

TABLE 10
Gel-cream experiment 1

| | Sample 1 | Sample 2 | Sample 3 | Sample 4 |
|---|---|---|---|---|
| Ingredient | % w/w | % w/w | % w/w | % w/w |
| Water | 88.5 | 88.5 | 88.5 | 88.5 |
| MHPC | 1.0 | 1.0 | 1.0 | 1.0 |
| CMC | 1.0 | 1.0 | 1.0 | 1.0 |
| Propylene glycol | 1.0 | 1.0 | 1.0 | 1.0 |
| 1.2-Hexanediol | 1.0 | 1.0 | 1.0 | 1.0 |
| Raspberry ketone | 0.5 | 0.5 | 0.5 | 0.5 |
| Sorbitan Oleate Decylglucoside | 5.5 | 5.0 | 3.36 | — |
| Crosspolymer (HLB 12-14) | | | | |
| Sorbitan Oleate Decylglucoside | — | — | 1.76 | 5.0 |
| Crosspolymer (HLB 8-10) | | | | |
| Avocado oil | 0.33 | 0.33 | 0.33 | 0.33 |
| Octyldodecyl Stearoyl Stearate | 0.67 | 0.67 | 0.67 | 0.67 |
| Fragrance mix | 1.0 | 1.0 | 1.0 | 1.0 |
| Appearance | translucent | translucent | opaque | translucent |
| Lum stability -index | 0.018 | 0.011 | 0.011 | 0.029 |
| Optic microscopy | Big oil | Smaller oil | Even smaller | Smallest but not |
| | droplets | droplets | droplet | homogeneous |

TABLE 11
Gel-cream- experiment 2

| | Sample 1 | | Sample 2 | |
|---|---|---|---|---|
| Ingredient | % w/w | | % w/w | |
| Water | 88.5 | | 85.5 | |
| MHPC | 1.0 | | 1.0 | |
| CMC | 1.0 | | 1.0 | |
| Propylene glycol | 1.0 | | 1.0 | |
| 1.2-Hexanediol | 1.0 | | 1.0 | |
| Raspberry Ketone | 0.5 | | 0.5 | |
| Sorbitan Oleate Decylglucoside | — | | — | |
| Crosspolymer (HLB 12-14) | | | | |
| Sorbitan Oleate Decylglucoside | 3.0 | | 6.0 | |
| Crosspolymer (HLB 8-10) | | | | |
| Avocado oil | 1.0 | | 1.0 | |
| Octyldodecyl Stearoyl Stearate | 2.0 | | 2.0 | |
| Fragrance mix | 1.0 | | 1.0 | |
| Appearance | white | | White | |
| Lum stability -index | no | | no | |
| Optic microscopy | yes | | yes | |
| Ph | 7.4 | | 7.2 | |
| Brookfield viscosity | 50000 | cps | 29000 | cps |
| | #95-20 | rpm | #95-20 | rpm |

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed! and/or claimed inventive concept(s).

What is claimed is:

1. An aqueous personal care composition comprising:
(a) 0.05 to 10 wt. % of low substituted carboxymethyl cellulose (CMC) alone or in combination with 0.01 to 10 wt. % of at least one natural polymer or synthetic polymer; and
(b) 0.01 to 60 wt. % of at least one personal care additive; and
(c) water;
wherein, the low substituted carboxymethyl cellulose has an average degree of carboxymethyl substitution (DS) per anhydroglucose unit is in the range of from about 0.5 to about <0.7.

2. The personal care composition according to claim 1, wherein the low substituted carboxymethyl cellulose has an average degree of carboxymethyl substitution (DS) per anhydroglucose unit is in the range of from about 0.5 to about 0.65.

3. The aqueous personal care composition according to claim 1, wherein the natural polymer is selected from the group consisting of xanthan gum, guar gum, carob gum, konjac gum, sclerotium gum, acacia gum, cellulose gum, pullulan, microcrystalline cellulose, Caesalpinia spinosa gum, carrageenan, dehydroxanthan gum, potato starch modified, glucomannan, magnesium aluminium silicate, bentonite, corn starch modified, polyester based polymer, tara gum, cassia gum, fenu greek, locust bean gum, pectine, starches and modified starches, alginate and alginic acid based polymers,, hemi-celluloses and gellan gum.

4. The aqueous personal care composition according to claim 1, wherein the synthetic polymer is an acrylate based polymer, methacrylate based polymer, maleic anhydride based polymer, polyurethane based polymer, vinyl pyrrolidone based polymer vinylcaprolactam based polymer, vinylalcohol based polymer, vinylamide based polymer, vinylether based polymers and polylactic acid/polylactic acid salt or modified polysaccharide polymer.

5. The aqueous personal care composition according to claim 4, wherein the modified polysaccharide polymer consisting of an methyl, hydroxyethyl, hydroxypropyl, carboxymethyl, sulfonate, phosphate, trimethylamine, hydroxypropyl, propyl groups of xanthan gum, guar gum, carob gum, konjac gum, sclerotium gum, acacia gum, cellulose gum, pullulan, microcrystalline cellulose (MCC), Caesalpinia spinosa gum, carrageenan, dehydroxanthan gum, potato starch modified, glucomannan, magnesium aluminium silicate, bentonite, corn starch modified, polyester based polymer, tara gum, cassia gum, fenu greek, locust bean gum, pectine, starches and modified starches, alginate and alginic acid based polymers, hemi-celluloses and gellan gum.

6. The aqueous personal care composition according to claim 1, wherein the low substituted CMC is in the form of solid particulate or powder.

7. The aqueous personal care composition according to claim 1, wherein the low substituted CMC has an average viscosity range of from about 2500 to about 20000 cps in 1% aqueous solution.

8. The aqueous personal care composition according to claim 1, wherein the low substituted CMC has a molecular weight in the range of from about 1000000 to about 2000000 Daltons.

9. The aqueous personal care composition according to claim 1, wherein the composition is an emulsion, a dispersion, a suspension, a lotion, a cream, a foam, a spray, a gel, a bar, a stick, a mask, a pad or a patch.

10. The aqueous personal care composition according to claim 1, wherein the personal care composition is formulated into a hair-care product, a shampoos, a hair conditioner, leave in and rinse off conditioner, a styling hair composition, a hair perming product, a hair relaxant, a hair straightener, a hair spray and lacquer, a permanent hair dyeing system, a hair styling mousse, a hair gel, a semi-permanent hair dyeing system, a temporary hair dyeing system, a hair bleaching system, a permanent hair wave system, a hair setting formulation, a liquid soap, a bar soap, a fragrance and/or odoriferous ingredients consisting preparation, a deodorizing and antiperspirant preparation, a body oil, a body lotion, a body gel, a treatment cream, a body cleaning product, a skin protection ointment, a shaving and aftershave preparation, a skin powder, a lipstick, a foundation, a nail varnish, an eye shadow, a mascara, a dry and moist make-up, a rouge, a powder, a depilatory agent, a leave-on skin lotion and cream, a shower gel, a toilet bar, a sunless tanner, a sunscreen lotion, a sunscreen spray, a sunscreen cream, a styling gel, a serum, a mask, a sunscreen lotion, dentifrice, and a tooth paste.

11. An aqueous hair care composition comprising:
(a) 0.05 to 10 wt. % of low substituted carboxymethyl cellulose (CMC) alone or in combination with 0.01 to 10 wt. % at least one natural or synthetic polymer;
(b) 0.01 to 60 wt. % at least one hair care additive; and
(c) water;
wherein, the low substituted carboxymethyl cellulose has an average degree of carboxymethyl substitution (DS) per anhydroglucose unit is in the range of from about 0.5 to about <0.7.

12. The personal care composition according to claim 11, wherein the low substituted carboxymethyl cellulose has an average degree of carboxymethyl substitution (DS) per anhydroglucose unit is in the range of from about 0.5 to about 0.65.

13. The aqueous hair care composition according to claim 11, wherein the natural or synthetic polymer is a dihydroxypropyl substituted cassia gum (DHP-CG polymer), a cationic guar, a hydroxyethyl cellulose (HEC), a methylhydroxypropyl cellulose (MHPC) or a sorbitan oleate decylglucoside cross-polymer.

14. The aqueous hair care composition according to claim 11, wherein hair care composition is formulated as a spray, a lotion, a mousse, a fluid, a serum, a solution, a perm, an emulsion, a gel, a vesicle a dispersion, a paste, a cream, a solid stick, a shampoo, a balm, a wipe, a milk, a foam, a jelly, a pomade, a wax, a powder, a bar or a liquid.

15. The aqueous hair care composition according to claim 11, wherein the hair care additive is selected from the group consisting of water-insoluble ingredients, oxidizing agents, conditioning agents, humectants, pH adjusting buffers, waxes, oils, emulsifiers, fragrances, preservatives, fatty substances, gelling agents, thickeners, emollients, hydrophilic or lipophilic active agent, antioxidants, sequestering agents, preserving agents, acidifying or basifying agents, fragrances, fillers, dyestuffs, plant extracts, moisturizers, proteins, peptides, neutralizing agents, solvents, anti-dandruff ingredients, reducing agents, silicones, ceraphyl esters and combinations thereof.

16. The aqueous hair care composition according to claim 15, wherein the hair care additives include oils selected from the group consisting of coconut oil, jojoba oil almond oil, olive oil, lavender oil, aloe vera oil, tea tree oil, grapeseed oil, safflower seed oil, argan oil, avocado oil, rosemary oil, cederwood oil, sage oil, arnica oil, eucalyptus oil, castor oil, thyme oil, rosemary oil, chamomile oil, macadamia nut oil, kukui nut oil, sunflower seed oil, sesame seed oil, rice bran oil, flax seed oil, soybean oil, pomegranate oil, peppermint oil, spearmint oil, clary sage oil and mixtures thereof.

17. The aqueous hair care composition according to claim 15, wherein the hair care additives consisting of silicones selected from the group consisting of silicone fluids (oils), silicone elastomers, silicone resins, polysiloxanes, linear silicones, branched silicones, cyclic silicones, aminosilicones, amodimethicone, dimethicones, dimethiconols, silicone polyethers, silicone quaternium compounds and mixtures thereof.

18. The aqueous hair care composition according to claim 11, wherein the composition can fix or treat hair imparting properties comprising detangling, wet combability, wet feel, dry combability, dry feel, sheen, static flyaway control, humidity resistance, hydrophobicity, surface smoothening, no build-up, color, high humidity curl retention, strong hold, and split end repair.

19. The aqueous hair care composition according to claim 11, wherein the pH of said hair care compositions is in the range of from about 3 to about 13.

20. The aqueous hair care composition according to claim 11, further comprises cosmetically acceptable excipient selected from the group consisting of fatty substances, gelling agents, thickeners, surfactants, moisturizers, emollients, hydrophilic or lipophilic active agent, antioxidants, sequestering agents, preserving agents, acidifying or basifying agents, fragrances, fillers, dyestuffs, emulsifying agents, solvents, UV-A or UV-B blocker/filters, plant extracts, moisturizers, proteins, peptides, neutralizing agents, solvents, silicones and reducing agents.
